(19) United States
(12) Patent Application Publication
(10) Pub. No.: US 2025/0090208 A1
(43) Pub. Date: Mar. 20, 2025

(54) PATIENT TRACKER SECURABLE TO BONE

(71) Applicants: Acclarent, Inc., Irvine, CA (US); Biosense Webster (Israel) Ltd., Yokneam (IL)

(72) Inventors: George Gusein, Acre (IL); Henry F. Salazar, Pico Rivera, CA (US); Cesar Fuentes-Ortega, Pasadena, CA (US); Hao Tran, Santa Ana, CA (US); Christopher A. Schutt, Bozeman, MT (US)

(21) Appl. No.: 18/883,152

(22) Filed: Sep. 12, 2024

Related U.S. Application Data
(60) Provisional application No. 63/539,344, filed on Sep. 20, 2023.

(51) Int. Cl. A61B 17/86; A61B 34/20

(52) U.S. Cl. CPC A61B 17/8605; A61B 2034/2055

(57) ABSTRACT

An apparatus includes a first body, a position sensor, and a screw. The first body defines a bore having a first length. The position sensor is fixed relative to the first body. The position sensor is configured to generate a signal indicating a real-time position of the position sensor within three-dimensional space. The screw is sized to fit in the bore and secure the first body to bone of a patient. The screw has a second length. The first length is greater than the second length.

This application claims priority under 35 U.S.C. § 119 to U.S. Patent Application Ser. No. 63/539,344, which was filed on Sep. 20, 2023 and is hereby incorporated herein by reference.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the TRUDI® Navigation System by Acclarent, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

One function that may be performed by an IGS system is obtaining one or more reference points that may be used to correlate various preoperatively obtained images with a patient's actual position during a procedure. This act may be referred to as patient registration. Such registration may be performed by using a positionally tracked instrument (e.g., a registration probe whose tip position may be detected in three-dimensional space) to trace or touch one or more positions on a patient's face. At each touch point, the IGS system may register that point in three-dimensional space; and, using a number of registered points, determine the position of the affected area in three-dimensional space. Once the affected area is fully mapped or registered, it may be correlated with preoperative images in order to provide a seamless IGS experience across varying types of preoperative images during the performance of the procedure.

While several systems and methods have been made and used in connection with IGS systems, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 is a schematic view of an example of an IGS system with a patient having their head turned to a side;

FIG. 2 is a perspective view of an example of a patient tracker that may be used with the IGS system of FIG. 1;

FIG. 3 is an exploded perspective view of the patent tracker of FIG. 2;

FIG. 4 is a perspective view of an example of a skin punch that may be used in a procedure with the patient tracker of FIG. 2;

FIG. 5 is a cross-sectional view of a head of a patient with the patient tracker of FIG. 2 secured to bone;

FIG. 6 is a cross-sectional side view of another example of a patient tracker that may be used with the IGS system of FIG. 1;

FIG. 7 is a perspective view of an anchor member of another example of a patient tracker that may be used with the IGS system of FIG. 1;

FIG. 8 is a top plan view of the anchor member of FIG. 7;

FIG. 9 is a cross-sectional side view of the anchor member of FIG. 7 taken along line 9-9 of FIG. 8, as viewed in the direction of the arrows;

FIG. 10 is a cross-sectional side view of a sensing member that may be combined with the anchor member of FIG. 7 to form a patient tracker; and

FIG. 11 is a cross-sectional side view of the anchor member of FIG. 7 and the sensing member of FIG. 10 combined to form a patient tracker.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

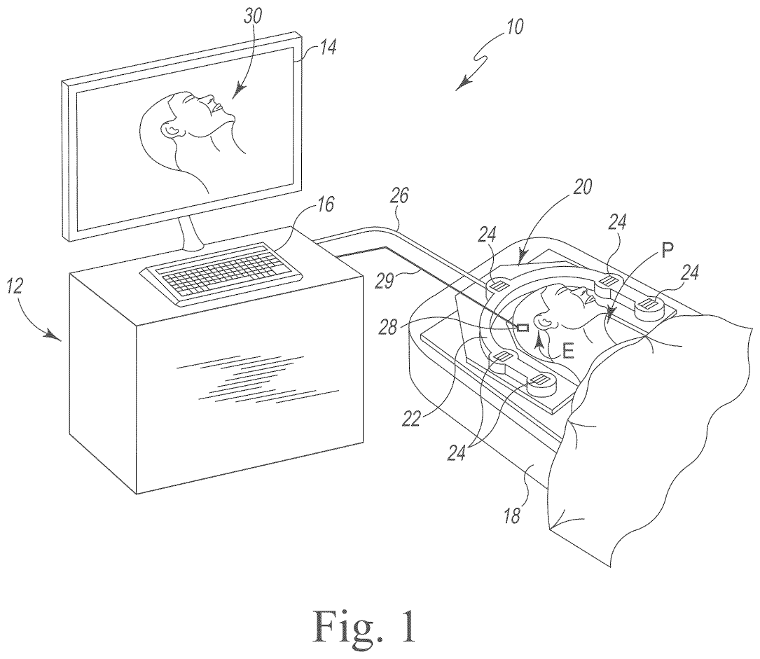

When performing a medical procedure within a head of a patient (P), it may be desirable to have information regarding the position of an instrument within the head of the patient (P), particularly when the instrument is in a location where it is difficult or impossible to obtain an endoscopic view of a working element of the instrument within the head of the patient (P). FIG. 1 shows an example of an IGS system 10 enabling a medical procedure to be performed within a head of a patient (P) using image guidance. In addition to or in lieu of having the components and operability described herein the IGS navigation system 10 may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety; and/or U.S. Pat. No. 10,561,370, entitled “Apparatus to Secure Field Generating Device to Chair,” issued Feb. 18, 2020, the disclosure of which is incorporated by reference herein, in its entirety.

The IGS system 10 of the present example includes a field generator assembly 20, which includes a set of magnetic field generators 24 that are integrated into a horseshoe-shaped frame 22. The field generators 24 are operable to generate alternating magnetic fields of different frequencies around the head of the patient (P). An instrument, such as any of the instruments described below, may be inserted into the head of the patient (P). Such an instrument may be a standalone device or may be positioned on an end effector. In the present example, the frame 22 is positioned on a table 18, with the patient (P) lying on their side on the table 18 such that the frame 42 is located adjacent to the head of the patient.

The IGS system 10 of the present example further includes a processor 12, which controls the field generators 24 and other elements of the IGS system 10. For instance, the processor 12 is operable to drive the field generators 24 to generate alternating electromagnetic fields; and process signals from the instrument to determine the location of a navigation sensor or position sensor in the instrument within the head of the patient (P). The processor 12 includes a processing unit (e.g., a set of electronic circuits arranged to evaluate and execute software instructions using combinational logic circuitry or other similar circuitry) communicating with one or more memories. The processor 12 is coupled with the field generator assembly 20 via a cable 26 in this example, though the processor 12 may alternatively be coupled with the field generator assembly 20 wirelessly or in any other suitable fashion.

A display screen 14 and user input feature 16 are also coupled with the processor 12 in this example. The user input feature 16 may include a keyboard, a mouse, a trackball, and/or any other suitable components, including combinations thereof. In some versions, the display screen 14 is in the form of a touchscreen that is operable to receive user inputs, such that the display screen 14 may effectively form at least part of the user input feature 160. A physician may use the input feature 16 to interact with the processor 12 while performing a registration process, while performing a medical procedure, and/or at other suitable times.

As described in greater detail below, a medical instrument may include a navigation sensor or position sensor that is responsive to positioning within the alternating magnetic fields generated by the field generators 24. In some versions, the navigation sensor or position sensor of the instrument may comprise at least one coil at or near the distal end of the instrument. When such a coil is positioned within an alternating electromagnetic field generated by the field generators 24, the alternating magnetic field may generate electrical current in the coil, and this electrical current may be communicated as position-indicative signals via wire or wirelessly to the processor 12. This phenomenon may enable the IGS system 10 to determine the location of the distal end of the instrument within a three-dimensional space (i.e., within the head of the patient (P), etc.). To accomplish this, the processor 12 executes an algorithm to calculate location coordinates of the distal end of the instrument from the position related signals of the coil(s) in the instrument. Thus, a navigation sensor may serve as a position sensor by generating signals indicating the real-time position of the sensor within three-dimensional space.

The processor 12 uses software stored in a memory of the processor 12 to calibrate and operate the IGS system 10. Such operation includes driving the field generators 24, processing data from the instrument, processing data from the user input feature 16, and driving the display screen 14. In some implementations, operation may also include monitoring and enforcement of one or more safety features or functions of the IGS system 10. The processor 12 is further operable to provide video and/or other images in real time via the display screen 14, showing the position of the distal end of the instrument in relation to a video camera image of the head of the patient (P), in relation to preoperative image (e.g., a CT scan image) of the head of the patient (P), and/or in relation to a computer-generated three-dimensional model of anatomical structures of the head of the patient (P). The display screen 14 may display such images simultaneously and/or superimposed on each other during the medical procedure. Such displayed images may also include graphical representations of instruments that are inserted in the head of the patient (P), or at least a position indicator (e.g., crosshairs, etc.), such that the operator may observe a visual indication of the instrument at its actual location in real time via the display screen 14.

In the example shown in FIG. 1, the display screen 14 is displaying a three-dimensional rendering 30 of the head of the patient (P). By way of further example only, the display screen 14 may provide images in accordance with at least some of the teachings of U.S. Pat. No. 10,463,242, entitled “Guidewire Navigation for Sinuplasty,” issued Nov. 5, 2019, the disclosure of which is incorporated by reference herein, in its entirety. In the event that the operator is also using an endoscope, the endoscopic image may also be provided on the display screen 14. The images provided through the display screen 14 may thus help guide the operator in maneuvering and otherwise manipulating instruments within the head of the patient (P).

In the present example, the field generators 24 are in fixed positions relative to the head of the patient (P), such that the frame of reference for the IGS system 10 (i.e., the electromagnetic field generated by field generators 24) does not move with the head of the patient (P). In some instances, the head of the patient (P) may not remain completely stationary relative to the field generators 24 throughout the duration of a medical procedure, such that it may be desirable to track movement of the head of the patient (P) during a medical procedure. To that end, the IGS system 10 of the present example includes a patient tracker 28 that is fixedly secured to the head of the patient (P). The Patient tracker 28 includes one or more coils and/or other position sensors that are operable to generate signals in response to the alternating magnetic fields generated by the field generators 24, with such signals indicating the position of the patient tracker 28 in three-dimensional space. In the present example, these signals are communicated to the processor 12 via a cable 29. In some other versions, these signals are communicated to the processor 12 wirelessly.

Regardless of how the processor 12 receives signals from the patient tracker 28, the processor 12 may utilize such signals to effectively track the real-time position of the head of the patient (P) and thereby account for any movement of the head of the patient (P) during a medical procedure. In other words, the processor 12 may process position-indicative signals from the patient tracker 28 in combination with position-indicative signals from a position sensor-equipped medical instrument that is disposed in the head of a patient (P) to accurately determine the real-time position of the distal end (or other working feature) of the medical instrument in the head of the patient (P) despite any movement of the head of the patient (P) during the medical procedure.

In the example shown in FIG. 1, the patient tracker 28 is positioned posterior to the ear (E) of the patient (P), though it should be understood that the patient tracker 28 may be positioned at any other suitable location on the head of the patient (P). By way of example only, the patient tracker 28 may alternatively be positioned at the lateral forehead, at the upper orbital rim, or at some other location near the site at which the medical procedure will be performed in the head of the patient (P). In some variations, the patient tracker 28 is positioned in the mouth of the patient (P). It should also be understood that the patient tracker 28 may be fixedly secured to the head of the patient (P) in numerous ways, including but not limited to adhesives, screws, tacks, sutures, etc. Further examples of how a tracking sensor such as the patient tracker 28 may be secured to the patient (P) will be described in greater detail below.

### II. Examples of Methods of Patient Trackers Securable to Bone

As noted above, it may be beneficial to track the real-time position of the head of a patient (P) via the patient tracker 28. Some versions of the patient tracker 28 may be secured to the skin of the patient (P), via an adhesive or otherwise. In some cases where the patient tracker 28 is secured to the skin of the patient (P), the position of the patent tracker 28 on the patient (P) may be unstable due to the elasticity or deformability of the skin. In cases where the position of the patient tracker 28 on the skin of the patient (P) shifts relative to the bone underlying the skin, the position data from the patient tracker 28 may be unreliable; and the shifting of the patient tracker 28 may cause shifting of the mapping/registration of the anatomy of the patient (P) with respect to IGS system 10. It may therefore be desirable to secure the patient tracker 28 directly to bone, to prevent the patient tracker 28 from shifting relative to anatomy of the patient (P) during a procedure in which the IGS system 10 is used.

In cases where the patient tracker 28 is secured directly to bone of a patient (P), it may also be desirable to minimize trauma to the bone and skin of the patient, such as by limiting the bone fixation to use of a single screw that is driven into the bone (rather than using two or more screws). Moreover, it may be desirable to protect the patient (P) from inadvertent slipping of a screwdriver from a screw as the screwdriver is being used to secure the patient tracker 28 to the patient (P) via the screw. The following describes examples of patient trackers that may provide one or more of the advantages described above.

### A. First Example of Patient Tracker

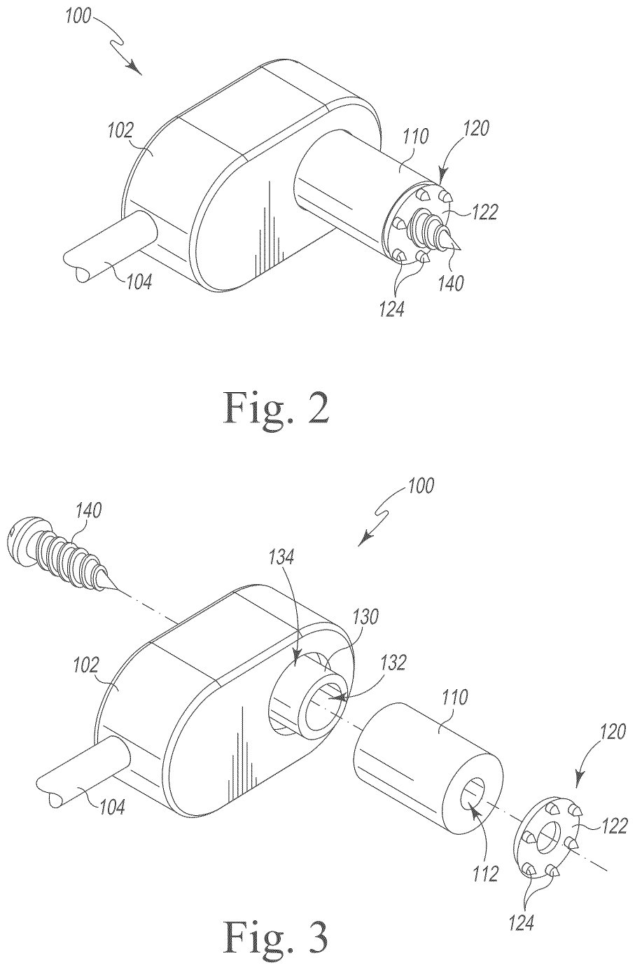

FIGS. 2-3 show an example of a patient tracker 100 that may be secured directly to bone of a patient (P). The patient tracker 100 of this example includes a body 102 defining a cylindraceous recess 134 around a mounting cylinder 130. The mounting cylinder 130 defines a bore 132. The body 102 of this example has an oblong shape, with the mounting cylinder 130 being positioned off-center, near a lateral end of the oblong shape.

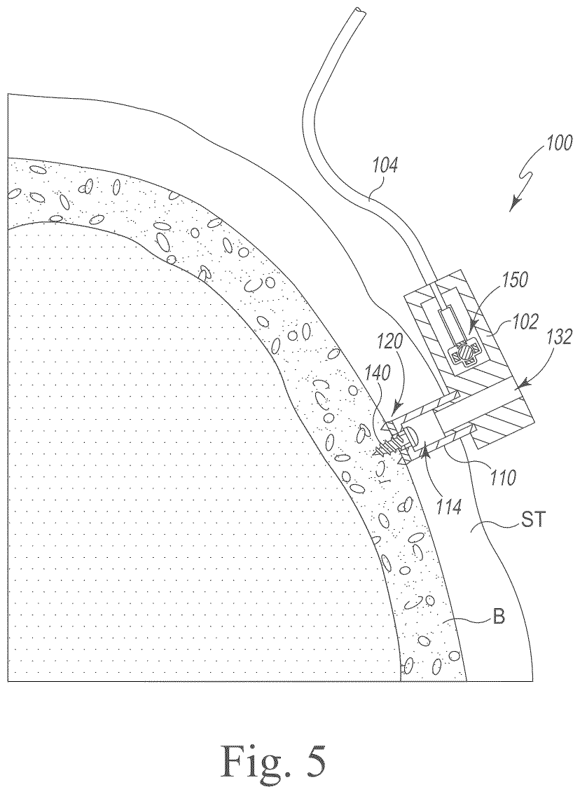

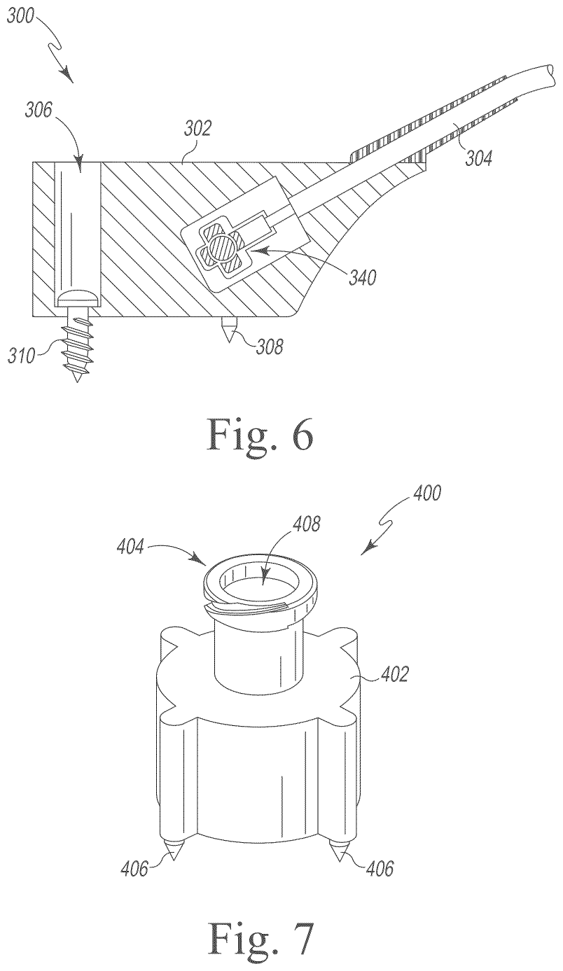

An outer cylinder 110 is fixedly mounted to the mounting cylinder 130 and defines a bore 114 (shown in FIG. 5) and an opening 112, with a proximal portion of the outer cylinder 110 being disposed within the cylindraceous recess 134. A traction member 120 comprises an annular body 122 that is fixedly secured to the distal end of the outer cylinder 110 (e.g., via an adhesive, via welding, etc.). A plurality of traction features 124 protrude from the body 122 of the traction member 120. In the present example, the traction features 124 are in the form of spikes, though the traction features 124 may have any other suitable form (e.g., teeth, friction-promoting surface treatment, etc.). The bores 114, 132 and the opening 112 are configured to align, such that the shaft of a screw 140 may pass through the bores 114, 132 and the opening 112; and further through the annular body 122.

While each bore 114, 132 has an inner diameter sized to accommodate the head of screw 140, the opening 112 has an inner diameter that is smaller than the head of the screw 140. In some versions, the bores 114, 132 together provide a combined bore length that is greater than the length of the screw 140. Thus, when the screw 140 is initially positioned within the bores 114, 132 and before the screw 140 is driven into bone as described below, the head of the screw 140 is already substantially recessed within the bores 114, 132.

As shown in FIG. 5, a position sensor 150 is positioned within the body 102. The position sensor 150 of this example comprises a plurality of coils. By way of further example only, the position sensor 150 may be in the form of a single-axis sensor, a dual-axis sensor, a tri-axis sensor (concentric or non-concentric), a plurality of scattered single-axis sensors, or take any other suitable form. When the coils of the position sensor 150 are placed within an alternating electromagnetic field generated by the field generators 24, the alternating magnetic field may generate electrical current in the coils of the position sensor 150, and this electrical current may be communicated as position-indicative signals via the cable 104 to the processor 12. This phenomenon may enable the IGS system 10 to determine the location of the patient tracker 100 within a three-dimensional space (i.e., within the head of the patient (P), etc.). While the position sensor 150 is communicatively coupled with the processor 12 via cable 104 in this example, the position sensor 150 may alternatively be communicatively coupled with the processor 12 wirelessly in some other versions.

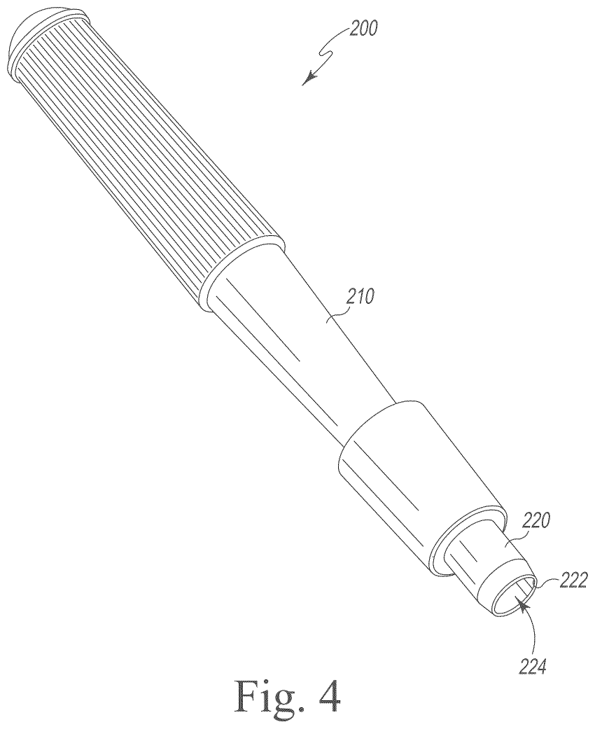

FIG. 4 shows an example of a skin punch 200 that may be used in a procedure where the patient tracker 100 is mounted to the head of a patient (P). The skin punch 200 of this example comprises a shaft 210 that is configured to be grasped by the hand of an operator. A cutting head 220 is positioned at the distal end of the shaft 210 and includes an annular cutting edge 222 that defines an opening 224. In the present example, the cutting head 220 has an outer diameter that is approximately equal to the outer diameter of the outer cylinder 110. By way of example only, this diameter may range from approximately 4 mm to approximately 6mm. Alternatively, the cutting head 220 and the outer cylinder 110 may have any other suitable outer diameter.

In an example of use of the skin punch 200 and the patient tracker 100, an operator may prepare the mounting site (e.g., by removing hair, etc.), grasp the skin punch 200 via the shaft 210, then press the cutting head 220 into the skin and other soft tissue until the cutting edge 222 reaches the underlying bone. The operator may then remove the skin punch 200 and scrape away any soft tissue that might remain on the bone, ultimately providing a circular region of exposed bone. The operator may then press the outer cylinder 110 into the passageway formed through the soft tissue by the skin punch 200 until the traction member 120 seats against the bone. The operator may then insert the screw 140 through the bores 114, 132, then insert a screwdriver head into the bores 114, 132, and drive the shaft of the screw 140 into the bone to thereby secure the patient tracker 100 to the bone via the screw 140. The resulting arrangement may appear similar to what is shown in FIG. 5. As shown, the outer cylinder 110 passes through the soft tissue (ST) (e.g., skin, etc.), with the screw 140 being driven into the the underlying bone (B). It should be understood that the positioning of the screwdriver head (not shown) in the bores 114, 132 while driving the screw 140 into the bone (B) may prevent the screwdriver head from inadvertently slipping from the head of the screw 140 and subsequently stabbing or scraping adjacent soft tissue (ST), particularly since the head of the screw 140 will be substantially recessed within the bores 114, 132 during the interaction between the screw 140 and the screwdriver. It should also be understood that, as the screw 140 is driven into the bone (B), the traction features 124 may embed in the bone (B) to prevent the patient tracker 100 from spinning about the axis shared by the screw 140 and the bores 114, 132; and may otherwise further stabilize the mount of the patient tracker 100 to the bone (B).

Since the body 102, the outer cylinder 110, the traction member 120, and the screw 140 are rigid in this example, since the position of the position sensor 150 is fixed relative to the body 102, and since the bone (B) to which the patient tracker 100 is mounted is rigid, the position data provided by the position sensor 150 may provide substantial reliability. In other words, the patient tracker 100 of the present example may provide substantially greater reliability than a patient tracker that is mounted to the skin of the head of the patient (P). Moreover, the configuration of the patient tracker 100 minimizes trauma to the patient (P) by providing only a single opening through soft tissue (ST) from the skin punch 200, by providing only a single screw 140 being inserted into bone (B), and by protecting the soft tissue (ST) from slippage of a screwdriver off of the screw 140 during driving of the screw 140 into bone (B).

In some variations, the outer cylinder 110 is threaded onto the mounting cylinder 130. This may allow the operator to adjust the position between the body 102 and the traction member 120, to accommodate different thicknesses of soft tissue (ST) over bone (B). For instance, the operator may provide relative rotation between the cylinders 110, 130 until the outer cylinder 110 and the traction member 120 protrude from the body 102 to a distance substantially equal to the thickness of the soft tissue (ST), such that the body 102 engages the exterior of the soft tissue (ST) when the patient tracker 100 is fully secured to the bone (B). In some cases, the threaded coupling may provide substantial friction that prevents inadvertent relative rotation between the cylinders 110, 130 after the patient tracker 100 is fully secured to the bone (B). Alternatively, the patient tracker 100 may provide any other suitable features to provide for adjustment to accommodate different thicknesses of soft tissue (ST) over bone (B) in different patients (P).

### B. Second Example of Patient Tracker

FIG. 7 shows another example of a patient tracker 300 that may be secured to bone of a patient (P). The patient tracker 300 of this example includes a rigid body 302 defining a bore 306 and housing a position sensor 340. The bore 306 is sized to receive a screw 310, similar to the bores 114, 132 described above. The bore 306 is also configured to contain the head of a screwdriver as the screwdriver is used to drive the screw 310 into bone, thereby preventing trauma that might otherwise result in the event that the screwdriver were to slip from the head of the screw 310. This may be due in part to the bore 306 having a length that is greater than the length of the screw 310. Thus, when the screw 310 is initially positioned within the bore 306 and before the screw 310 is driven into bone as described herein, the head of the screw 310 is already substantially recessed within the bore 306.

The position sensor 340 is configured and operable like the position sensor 150 described above. Thus, the position sensor 340 of this example includes a plurality of coils. By way of further example only, the position sensor 340 may be in the form of a single-axis sensor, a dual-axis sensor, a tri-axis sensor (concentric or non-concentric), a plurality of scattered single-axis sensors, or take any other suitable form. When the coils of the position sensor 340 are placed within an alternating electromagnetic field generated by the field generators 24, the alternating magnetic field may generate electrical current in the coils of the position sensor 340, and this electrical current may be communicated as position-indicative signals via the cable 304 to the processor 12. This phenomenon may enable the IGS system 10 to determine the location of the patient tracker 300 within a three-dimensional space (i.e., within the head of the patient (P), etc.). While the position sensor 340 is communicatively coupled with the processor 12 via the cable 304 in this example, the position sensor 340 may alternatively be communicatively coupled with the processor 12 wirelessly in some other versions.

The patient tracker 300 of this example further includes a traction feature 308 in the form of a spike protruding downwardly from the body 302, though it should be understood that the traction feature 308 may take any other suitable form (e.g., teeth, friction-promoting surface treatment, etc.). While only one traction feature 308 is shown, some other versions may provide two or more traction features 308. It should also be understood that such traction feature(s) 308 may be positioned at any suitable location(s) along the body 302. In some versions, the traction feature 308 is configured to engage skin or other soft tissue of the patient (P) while the screw 310 is driven into the bone of the patient (P). In some such cases, some soft tissue is cleared away from the bone to provide clearance for the screw 310 to be driven into the bone, but additional soft tissue is not cleared away for the traction feature 308 to directly engage bone. In some other cases, additional soft tissue is cleared away to allow the traction feature 308 to directly engage bone. In either scenario, the traction feature 308 may substantially prevent the body 302 from rotating about the spinning about the axis shared by the screw 310 and the bore 306; and may otherwise further stabilize the mount of the patient tracker 300 to the patient (P).

### C. Third Example of Patient Tracker

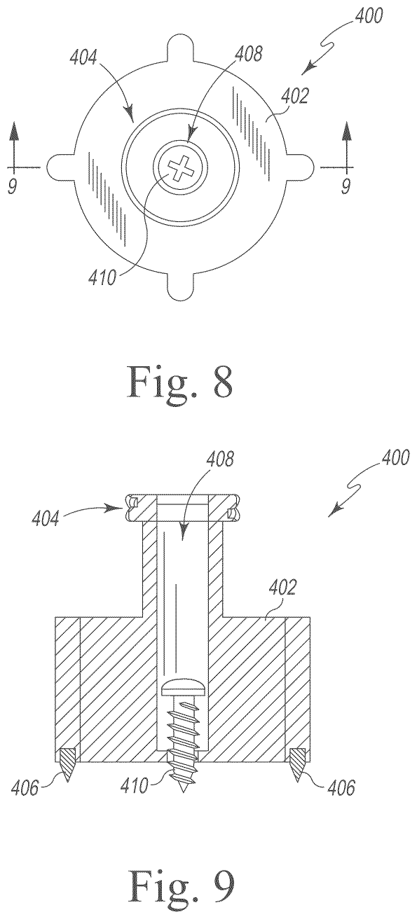

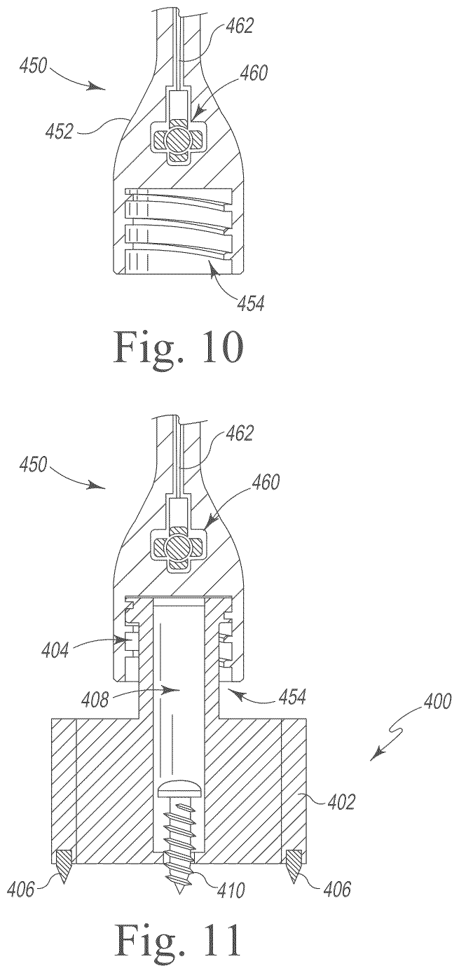

FIGS. 7-11 show other features that may be used to form a patient tracker. In particular, FIGS. 7-9 show an anchor member 400, FIG. 10 shows a sensing member 450, and FIG. 11 shows the sensing member 450 coupled with the anchor member 400 to form a patient tracker. The anchor member 400 of this example includes a rigid body 402 that includes a male luer feature 404 (i.e., luer lock or luer fitting), a set of traction features 406, and a central bore 408. The bore 408 is sized to receive a screw 410, similar to the bores 114, 132, 306 described above. The bore 408 is also configured to contain the head of a screwdriver as the screwdriver is used to drive the screw 410 into bone, thereby preventing trauma that might otherwise result in the event that the screwdriver were to slip from the head of the screw 410. This may be due in part to the bore 408 having a length that is greater than the length of the screw 410. Thus, when the screw 410 is initially positioned within the bore 408 and before the screw 410 is driven into bone as described herein, the head of the screw 410 is already substantially recessed within the bore 408. In the present example, the male luer feature 404 is coaxial with the bore 408.

The traction features 406 of this example are in the form of spikes protruding downwardly from the body 402, though it should be understood that the traction features 406 may take any other suitable form (e.g., teeth, friction-promoting surface treatment, etc.). While the anchor member 400 has four traction features 406 in this example, some other versions may provide more or fewer than four traction features 406. In addition, while the traction features 406 are positioned along the outer periphery of the body 402 in this example, in an angularly spaced array about the bore 408, it should be understood that the traction features 406 may be positioned at any suitable locations along the body 406. In some versions, each of the traction features 406 is configured to engage skin or other soft tissue of the patient (P) while the screw 410 is driven into the bone of the patient (P). In some such cases, some soft tissue is cleared away from the bone to provide clearance for the screw 410 to be driven into the bone, but additional soft tissue is not cleared away for the traction features 406 to directly engage bone. In some other cases, additional soft tissue is cleared away to allow the traction features 406 to directly engage bone. In either scenario, the traction features 406 may substantially prevent the body 402 from rotating about the spinning about the axis shared by the screw 410 and the bore 408; and may otherwise further stabilize the mount of the patient tracker 400 to the patient (P).

As shown in FIG. 10, the sensing member 450 of the present example includes a rigid body 452 defining a female luer feature 454 and housing a position sensor 460. In the present example, the position sensor 460 is positioned along a shared axis with the female luer feature 454. The position sensor 460 is configured and operable like the position sensors 150, 340 described above. Thus, the position sensor 460 of this example includes a plurality of coils. By way of further example only, the position sensor 460 may be in the form of a single-axis sensor, a dual-axis sensor, a tri-axis sensor (concentric or non-concentric), a plurality of scattered single-axis sensors, or take any other suitable form. When the coils of the position sensor 460 are placed within an alternating electromagnetic field generated by the field generators 24, the alternating magnetic field may generate electrical current in the coils of the position sensor 460, and this electrical current may be communicated as position-indicative signals via the cable 462 to the processor 12. This phenomenon may enable the IGS system 10 to determine the location of the sensing member 450 within a three-dimensional space (i.e., within the head of the patient (P), etc.). While the position sensor 460 is communicatively coupled with the processor 12 via the cable 462 in this example, the position sensor 460 may alternatively be communicatively coupled with the processor 12 wirelessly in some other versions.

As shown in FIG. 11, the anchor member 400 and the sensing member 450 may be coupled together via complementary luer features 404, 454. In particular, the operator may first mount the anchor member 400 to bone via the screw 410, before the sensing member 450 is coupled with the anchor member 400. Once the anchor member 400 has been suitably secured to the bone of the patient (P), the operator may insert the male luer feature 404 of the anchor member 400 into the female luer feature 454 of the sensing member 450, then press the sensing member 450 toward the anchor member 400 while twisting the sensing member 450 relative to the anchor member 400 to thereby secure the sensing member 450 to the anchor member via the luer features 404, 454. At this stage, with the anchor member 400 firmly mounted to the bone of the patient (P) via the screw 410, and with the sensing member 450 firmly mounted to the anchor member 400 via the luer features 404, 454, the location of the position sensor 460 relative to the bone of the patient (P) may remain substantially fixed, thereby providing substantially reliable tracking of the real-time position of the patient (P) in three-dimensional space. As shown in FIG. 11, the coupling of the sensing member 450 with the anchor member 400 will effectively close off the proximal end of the bore 408, thereby enclosing the screw 410.

In some scenarios, the anchor member 400 may be provided as a single-use component, while the sensing member 450 may be provided as a reusable component. Such an arrangement may minimize waste and expense.

While screws the 140, 310, 410 are used to secure the patient tracker 100, 300 or anchor member 400 to bone in this example, in some versions at least a portion of the body 102, 302, 402 may include an adhesive that further secures the body 102, 302, 402 relative to skin. Such an adhesive may facilitate mounting of the patient tracker 100, 300 or the anchor member 400 to bone, though such an adhesive may not necessarily contribute to the fixation of the patient tracker 100, 300 or anchor member 400 relative to the patient once the screw 140, 310, 410 has been sufficiently installed.

In some cases, the patient tracker 100, 300 or anchor member 400 is secured to bone (B) on the lateral side of the head of the patient (P) (e.g., to the temporal bone). In some other cases, the patient tracker 100, 300 or anchor member 400 is secured to bone on the anterior or posterior side of the head of the patient (P). It should therefore be understood that the patient tracker 100, 300 or anchor member 400 may be secured to any suitable location on the patient (P). The patient tracker 100, 300 or the anchor member 400 and the sensing member 450 may be used in various kinds of medical procedures, including but not limited to ear, nose, and throat (ENT) procedures, cranial procedures, otology procedures, spinal procedures, neurosurgery procedures where a craniotomy is required, neurotology procedures, etc.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a first body, the first body defining a bore, the bore having a first length; (b) a position sensor fixed relative to the first body, the position sensor being configured to generate a signal indicating a real-time position of the position sensor within three-dimensional space; and (c) a screw, the screw being sized to fit in the bore and secure the first body to bone of a patient, the screw having a second length, the first length being greater than the second length.

### Example 2

The apparatus of Example 1, the first body having an oblong shape.

### Example 3

The apparatus of Example 2, the bore being positioned off-center, near a lateral end of the oblong shape.

### Example 4.

The apparatus of any of Examples 1 through 3, the first body further comprising: (i) a mounting cylinder defining the bore, and (ii) a cylindraceous recess around a proximal end of the mounting cylinder.

### Example 5

The apparatus of Example 4, further comprising an outer cylinder mounted to the mounting cylinder.

### Example 6

The apparatus of Example 5, the outer cylinder having an opening with an inner diameter, the screw having a head with a head outer diameter and a shaft with a shaft outer diameter, the inner diameter of the opening being smaller than the head outer diameter but at least as large as the shaft outer diameter.

### Example 7

The apparatus of any of Examples 1 through 6, further comprising a traction feature, the traction feature being configured to engage bone or tissue of a patient and thereby prevent the first body from rotating about an axis of the bore.

### Example 8

The apparatus of Example 7, the traction feature comprising one or more spikes.

### Example 9

The apparatus of any of Examples 7 through 8, the traction feature extending integrally from the first body.

### Example 10

The apparatus of any of Examples 7 through 8, further comprising a cylinder extending from the first body, the traction feature extending integrally from the cylinder.

### Example 11

The apparatus of any of Examples 1 through 10, further comprising a cable extending from the first body, the cable being configured to communicate signals from the position sensor to a processor.

### Example 12

The apparatus of any of Examples 1 through 11, further comprising a second body, the position sensor being positioned within the second body.

### Example 13

The apparatus of Example 12, the second body being fixedly secured to the first body by complementary coupling features.

### Example 14

The apparatus of Example 13, the complementary coupling features comprising luer lock features.

### Example 15

The apparatus of any of Examples 1 through 14, the first body defining a central axis, the bore extending along the central axis.

### Example 16

A method comprising: (a) forming an opening in soft tissue of a patient; (b) positioning a first body of a patient tracker against bone of the patient, the bone being exposed through the opening; (c) placing a tip of a screw in initial contact with the bone, the screw being disposed in a bore of the first body, the screw having a head that is recessed within the bore during the act of placing the tip of the screw in initial contact with the bone; (d) inserting a head of a screwdriver into the bore, such that the head of the screwdriver is recessed within the bore, and such that the head of the screwdriver engages the head of the screw; (e) driving the screw into the bone via the screwdriver, thereby fixedly securing the first body of the patient tracker to the bone; and (f) receiving a signal from a position sensor of the patient tracker after the patient tracker is fixedly secured to the bone, the signal indicating a real-time position of the patient tracker in three-dimensional space.

### Example 17

The method of Example 16, the act of forming an opening in soft tissue comprising pressing a skin punch through the soft tissue, the skin punch having an annular distal cutting edge.

### Example 18

The method of any of Examples 16 through 17, further comprising engaging one or more traction features of the patient with tissue of the patient, the one or more traction features being configured to resist rotation of the first body about an axis.

### Example 19

The method of Example 18, the one or more traction features engaging bone tissue.

### Example 20

The method of any of Examples 18 through 19, the one or more traction features comprising one or more spikes.

### Example 21

The method of any of Examples 16 through 20, the soft tissue and the bone being located on a head of the patient.

### Example 22

The method of any of Examples 16 through 20, further comprising adjusting a configuration of the patient tracker based on a thickness of the soft tissue.

### Example 23

The method of Example 22, the patient tracker further comprising a cylinder, the act of adjusting a configuration of the patient tracker based on a thickness of the soft tissue comprising moving the cylinder relative to the first body.

### Example 24

The method of Example 23, the cylinder being threadably coupled with the first body, the act of moving the cylinder relative to the first body comprising rotating the cylinder relative to the first body.

### Example 25

The method of any of Examples 16 through 24, the position sensor being contained within the first body.

### Example 26

The method of any of Examples 16 through 24, the position sensor being contained within a second body, the method further comprising coupling the second body with the first body.

### Example 27

The method of Example 26, the first body comprising a first luer feature, the second body comprising a second luer feature, the act of coupling the second body with the first body comprising coupling the first luer feature with the second luer feature.

### Example 28

The method of any of Examples 26 through 27, the second body covering an open proximal end of the bore after the second body is coupled with the first body, thereby covering the head of the screw.

### Example 29

The method of any of Examples 16 through 28, further comprising: (a) inserting a working instrument into the patient while the first body of the patient tracker is fixedly secured to the bone, the working instrument including a position sensor; (b) tracking a real-time position of the working instrument within the patient within three-dimensional space, based on signals from the position sensor of the working instrument, while simultaneously tracking a real-time position of the patient within three-dimensional space, based on signals from the position sensor of the patient tracker.

### Example 30

An apparatus comprising: (a) an anchor member, the anchor member including: (i) a first body defining a bore, the bore being configured to receive a screw, (ii) a first coupling feature, and (iii) one or more traction features configured to engage tissue of a patient; and (b) a sensing member, the sensing member including: (i) second body, (ii) a position sensor secured within the second body, the position sensor being configured to generate signals indicating a real-time position of the sensing member in three-dimensional space, and (iii) a second coupling feature, the second coupling feature being configured to removably couple with the first coupling feature to thereby removably couple the sensing member with the anchor member.

### Example 31

The apparatus of Example 30, further comprising a screw, the bore having a first length, the screw having a second length, the second length being shorter than the first length.

### Example 32

The apparatus of any of Examples 30 through 31, the first coupling feature being coaxially positioned with the bore.

### Example 33

The apparatus of any of Examples 30 through 32, the first coupling feature comprising a luer feature.

### Example 34

The apparatus of any of Examples 30 through 33, the one or more traction features comprising one or more spikes.

### Example 35

The apparatus of any of Examples 30 through 34, the one or more traction features being positioned in an angularly spaced array on the body about the bore.

### Example 36

The apparatus of any of Examples 30 through 35, the position sensor being positioned along a shared axis with the second coupling feature.

### Example 37

The apparatus of any of Examples 30 through 36, the sensing member being configured to close a proximal end of the bore when the second coupling feature is coupled with the first coupling feature.

### Example 38

A method comprising: (a) placing a tip of a screw in initial contact with bone of a patient, the screw being disposed in a bore of an anchor member body of a patient tracker; (b) inserting a head of a screwdriver into the bore, such that the head of the screwdriver engages the head of the screw; (c) driving the screw into the bone via the screwdriver, thereby fixedly securing the anchor member of the patient tracker to the bone; (d) securing a sensing member of the patient tracker to the anchor member of the patient tracker after the anchor member of the patient tracker is fixedly secured to the bone; and (e) receiving a signal from a position sensor of the sensing member of the patient tracker after the sensing member of the patient tracker is fixedly secured to the anchor member of the patient tracker, the signal indicating a real-time position of the patient tracker in three-dimensional space.

### Example 39

The method of Example 38, the screw having a head that is recessed within the bore during the act of placing the tip of the screw in initial contact with the bone, the head of the screwdriver being recessed in the bore upon inserting the head of the screwdriver into the bore to engage the head of the screw.

### Example 40

The method of any of Examples 38 through 39, further removing soft tissue from the bone to thereby expose the bone before placing the tip of the screw in initial contact with bone.

### Example 41

The method of any of Examples 38 through 40, further comprising: (a) placing the anchor member body against tissue of the patient; and (b) engaging the tissue of the patient with one or more traction features of the anchor member body.

### Example 42

The method of Example 41, the tissue comprising bone tissue.

### Example 43

The method of any of Examples 38 through 42, the act of securing a sensing member of the patient tracker to the anchor member of the patient tracker comprising aligning a coupling feature of the sensing member of the patient tracker with a coupling feature of the anchor body of the patient tracker and with the bore, such that the coupling feature of the sensing member of the patient tracker, the coupling feature of the anchor body of the patient tracker, and the bore are coaxially aligned.

### Example 44

The method of any of Examples 38 through 43, the act of securing the sensing member of the patient tracker to the anchor member of the patient tracker comprising pressing the sensing member of the patient tracker against the anchor member of the patient tracker while rotating the sensing member of the patient tracker relative to the anchor member of the patient tracker.

### Example 45

The method of any of Examples 38 through 44, the act of securing the sensing member of the patient tracker to the anchor member of the patient tracker resulting in enclosure of the screw within the bore, due to the sensing member of the patient tracker covering the proximal end of the bore.

### IV. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An apparatus comprising:
a first body, the first body defining a bore, the bore having a first length;
a position sensor fixed relative to the first body, the position sensor being configured to generate a signal indicating a real-time position of the position sensor within three-dimensional space; and
a screw, the screw being sized to fit in the bore and secure the first body to bone of a patient, the screw having a second length, the first length being greater than the second length.

2. The apparatus of claim 1, wherein the first body has an oblong shape.

3. The apparatus of claim 2, wherein the bore is positioned off-center, near a lateral end of the oblong shape.

4. The apparatus of claim 1, wherein the first body further comprises:
a mounting cylinder defining the bore, and
a cylindraceous recess around a proximal end of the mounting cylinder.

5. The apparatus of claim 4, further comprising an outer cylinder mounted to the mounting cylinder.

6. The apparatus of claim 5, wherein:
the outer cylinder has an opening with an inner diameter,
the screw has a head with a head outer diameter and a shaft with a shaft outer diameter, and
the inner diameter of the opening is smaller than the head outer diameter but at least as large as the shaft outer diameter.

7. The apparatus of claim 1, further comprising a traction feature, wherein the traction feature is configured to engage bone or tissue of a patient and thereby prevent the first body from rotating about an axis of the bore.

8. The apparatus of claim 7, wherein the traction feature comprises one or more spikes.

9. The apparatus of claim 7, wherein the traction feature extends integrally from the first body.

10. The apparatus of claim 7, further comprising a cylinder extending from the first body, wherein the traction feature extends integrally from the cylinder.

11. The apparatus of claim 1, further comprising a cable extending from the first body, wherein the cable is configured to communicate signals from the position sensor to a processor.

12. The apparatus of claim 1, further comprising a second body, wherein the position sensor is positioned within the second body.

13. The apparatus of claim 12, wherein the second body is fixedly secured to the first body by complementary coupling features.

14. The apparatus of claim 13, wherein the complementary coupling features comprise luer lock features.

15. The apparatus of claim 1, wherein the first body defines a central axis, wherein the bore extends along the central axis.

16. A method comprising:
forming an opening in soft tissue of a patient;
positioning a first body of a patient tracker against bone of the patient, the bone being exposed through the opening;
placing a tip of a screw in initial contact with the bone, the screw being disposed in a bore of the first body, the screw having a head that is recessed within the bore during the act of placing the tip of the screw in initial contact with the bone;
inserting a head of a screwdriver into the bore, such that the head of the screwdriver is recessed within the bore, and such that the head of the screwdriver engages the head of the screw;
driving the screw into the bone via the screwdriver, thereby fixedly securing the first body of the patient tracker to the bone; and
receiving a signal from a position sensor of the patient tracker after the patient tracker is fixedly secured to the bone, the signal indicating a real-time position of the patient tracker in three-dimensional space.

17. The method of claim 16, wherein forming an opening in soft tissue comprises pressing a skin punch through the soft tissue, the skin punch having an annular distal cutting edge.

18. The method of claim 16, further comprising engaging one or more traction features with tissue of the patient, the one or more traction features being configured to resist rotation of the first body about an axis.

19. The method of claim 18, wherein the one or more traction features engage bone tissue.

20. The method of claim 18, wherein the one or more traction features comprise one or more spikes.

21. The method of claim 16, wherein the soft tissue and the bone are located on a head of the patient.

22. The method of claim 16, further comprising adjusting a configuration of the patient tracker based on a thickness of the soft tissue.

23. The method of claim 22, wherein:
the patient tracker further comprises a cylinder, and adjusting a configuration of the patient tracker based on a thickness of
the soft tissue comprises moving the cylinder relative to the first body.

24. The method of claim 23, wherein:
the cylinder is threadably coupled with the first body, and
moving the cylinder relative to the first body comprises rotating the cylinder relative to the first body.

25. The method of claim 16, wherein the position sensor is contained within the first body.

26. The method of claim 16, wherein the position sensor is contained within a second body, further comprising coupling the second body with the first body.

27. The method of claim 26, wherein:
the first body comprises a first luer feature,
the second body comprises a second luer feature, and
coupling the second body with the first body comprises coupling the first luer feature with the second luer feature.

28. The method of claim 26, wherein the second body covers an open proximal end of the bore after the second body is coupled with the first body, thereby covering the head of the screw.

29. The method of claim 16, further comprising:
inserting a working instrument into the patient while the first body of the patient tracker is fixedly secured to the bone, the working instrument including a position sensor; and
tracking a real-time position of the working instrument within the patient within three-dimensional space, based on signals from the position sensor of the working instrument, while simultaneously tracking a real-time position of the patient within three-dimensional space, based on signals from the position sensor of the patient tracker.
